SEQUENCE LISTING (2 sequences)

SEQ ID NO: 1 - DNA, 21 bp, Artificial Sequence
  SEQ ID NO1 - Forward PCR Primer nptII
gaggctattcggctatgactg

SEQ ID NO: 2 - DNA, 21 bp, Artificial Sequence
  SEQ ID NO2 - Reverse PCR Primer nptII
atcgggagcggcgataccgta

(19) United States
(12) Patent Application Publication
(10) Pub. No.: US 2022/0041977 A1
(43) Pub. Date: Feb. 10, 2022

(54) MATRIX-MEDIATED CELL CULTURE SYSTEM

(71) Applicant: University of Cape Town, Cape Town (ZA)

(72) Inventors: Dylan Troy STEVENS, Cape Town (ZA); Edward Peter RYBICKI, Cape Town (ZA); Susan T L HARRISON, Cape Town (ZA)

(73) Assignee: University of Cape Town, Cape Town (ZA)

(21) Appl. No.: 17/298,059

(22) PCT Filed: Nov. 29, 2019
(86) PCT No.: PCT/IB2019/060330

§ 371 Date: May 28, 2021

(30) Foreign Application Priority Data
Nov. 29, 2018 (ZA) 2018/08076

(51) Int. Cl. C12N 1/12; C12N 1/04; C12N 5/00; C12N 5/14

(52) U.S. Cl. CPC C12N 1/12; C12N 1/04; C12N 5/0025; C12R 2001/89; C12N 2533/10; C12N 2513/00; C12N 2799/02; C12N 5/14

(57) ABSTRACT

The invention relates to a matrix-mediated algal cell culture system comprising a porous matrix, a microalgal cell culture comprising cells immobilised on the porous matrix, and a vector including a nucleic acid sequence encoding a heterologous polypeptide of interest, wherein immobilisation of microalgal cells on the porous matrix results in the formation of interstitial spaces between the microalgal cells to allow for increased contact of the microalgal cells with the vector compared with a culture of microalgal cells which are not immobilised on a porous matrix, thereby allowing for more efficient transfection of the microalgal cells with the vector. The invention also relates to methods of screening single species of microalgae and mixed ecology samples for the ability to be transfected using the algal cell culture system, and to methods for the production of heterologous polypeptides using the matrix-mediated cell culture system.

### BACKGROUND OF THE INVENTION

The present invention relates to a matrix-mediated cell harvesting and cell maintenance system, methods of screening single species of microalgae and mixed ecology samples for the ability to be transfected using the matrix-mediated cell culture system, as well as methods of producing heterologous polypeptides using the matrix-mediated cell culture system.

Plant cell cultures have several advantages as a production platform for heterologous proteins compared to more traditional platforms. These advantages include sterile culture environments, uniform cell types being used for production, and volumetric production in liquid bioreactors rather than in a 2D fashion as is the case with whole plants. This allows for complete cellular containment within ISO- and GMP-certified bioreactors, with ease of sterility maintenance as well as strain maintenance, which is not possible with whole plants.

Microalgae are also good candidates for heterologous protein production, and have several added advantages over plant cells. Firstly, many species are able to be grown in the dark, like cultured plant cells, utilizing a carbon source for energy requirements, and so are able to be cultivated in industrial bioreactors. However, microalgae require much simpler and cheaper media for reproduction than plant cell cultures. To add to this, many microalgae, unlike plant cell cultures, can be cryogenically preserved indefinitely. This is a sizeable advantage, as algal isolates that have been identified to have industrial application can be evolutionarily halted via cryogenesis, ensuring a high degree of consistency and low batch variation in terms of heterologous protein production, among other possible applications.

In addition, microalgae have the added advantage that they are naturally single-celled, unlike plant cell cultures that must be synthetically manipulated to grow in this state. As such microalgae have several associated advantages over synthetic plant cell cultures. These include not requiring complex plant hormones for culturing, and most importantly, they can be screened for industrial applicability a priori. This is due to the fact that a single microalgal cell possesses the biochemical and genomic potential of the entire species, so that numerous species can be screened simultaneously for a specific trait, such as industrial applicability. This is in stark contrast to plant cell cultures where a plant is chosen and induced into this single-celled state, making high throughput selection of different species impossible.

Further, currently known plant cell-packs are highly inefficient when larger pack volumes are used. This is due to two conceptual shortfalls. First, as the cell pack gets larger, the plant cells act as a filter, so filtering out A. tumefaciens. Secondly, although plant cells vary in size across species, they are the same size in a culture. This means that as the cell-pack gets larger, which is fundamental for industrial implementation of the process, the additive effects of a constant cell size culminate in a capping or limiting of possible pack size. Cells in plant cell cultures also tend to adhere to one another, which results in low transformation efficiency of the cells due to reduced contact with the transformation vector. A further advantage of using algal cell cultures is that it is easier to culture algal cells

However, a drawback of using microalgae cell cultures is that microalgae have low transfection efficiency in culture, partly due to the small size of algal cells and thus reduced contact between the algal cells and Agrobacterium spp. in culture. It is thus an aim of the present invention to provide a microalgae cell culture system while at the same time increasing the transfection efficiency of the microalgae.

### SUMMARY OF THE INVENTION

The invention relates to a matrix-mediated algal cell culture system, methods of screening single species of microalgae and mixed ecology samples for the ability to be transfected using the algal cell culture system, and to methods for the production of heterologous polypeptides using the matrix-mediated cell culture system.

According to a first aspect of the invention there is provided for a matrix-mediated cell culture system comprising (i) a porous matrix; (ii) a microalgal cell culture comprising microalgal cells immobilised on the porous matrix; and (iii) a vector including a nucleic acid sequence encoding a heterologous polypeptide of interest, wherein the immobilisation of the microalgal cells on the porous matrix results in the formation of interstitial spaces between the microalgal cells to allow for increased contact of the microalgal cells with the vector compared with a culture of microalgae cells which are not immobilised on a porous matrix, thereby allowing the microalgal cells to be transfected with the vector.

In a first embodiment of the present invention the porous matrix may comprise diatomaceous earth (SiO₂), although those of skill in the art will appreciate that other porous materials could be used. Preferably the matrix material has a particle size in the same range as the size of the microalgal cells. For example, the particle size of the porous material may range in size from 1 μm to 1000 μm. It will be appreciated by those of the skill in the art that microalgae range in size from 1 μm to 1000 μm. Therefore, the particle size of the porous material may also be from about 5 μm to about 900 μm, such as about 5 μm, about 10 μm, about 15 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 200 μm, about 300 μm, about 400 μm, about 500 μm, about 600 μm, about 700 μm, about 800 μm or about 900 μm.

According to a second embodiment of the present invention the vector may be an Agrobacterium spp. vector.

In a third embodiment of the invention the heterologous polypeptide of interest is a reporter polypeptide, preferably a reporter polypeptide selected from the group consisting of luciferase, alkaline phosphatase, green fluorescent protein, beta-galactosidase, horse radish peroxidase, and β-glucuronidase.

According to a fourth embodiment the heterologous polypeptide of interest is a pharmacological polypeptide.

In a further embodiment of the present invention, the heterologous polypeptide of interest is exported or secreted from the microalgal cell into the medium and recovered from the medium.

In another embodiment the microalgal cell culture may comprise an axenic sample comprising a single species of microalgae or a mixed ecology sample comprising a plurality of microalgae species. It will be appreciated that the mixed ecology sample may further comprise spores, bacteria, zooplankton and/or macroalgae.

In a further embodiment of the present invention the matrix-mediated cell culture system may be medium-deprived.

In yet another embodiment, the immobilisation of the microalgal cells on the porous matrix is by non-covalent adhesion of the microalgal cells to the porous matrix.

According to a second aspect of the present invention there is provided for a method of screening a species of microalgae for its ability to be transfected by a vector, the method comprising (i) providing the matrix-mediated cell culture system of the first aspect as described herein; (ii) incubating the immobilised microalgal cells with the vector; (iii) transfecting the microalgal cells with the vector; and (iv) detecting expression of the heterologous polypeptide of interest, wherein expression of the heterologous polypeptide of interest is indicative of the ability of the species of microalgae to be transfected.

In a first embodiment of the method of screening a species of microalgae for its ability to be transfected by a vector the microalgal cell culture may comprise an axenic sample comprising a single species of microalgae.

In a second embodiment of the method of screening a species of microalgae for its ability to be transfected by a vector, the method further comprises a step of removing medium from the immobilised microalgal cells prior to the step of incubating the immobilised microalgal cells with the vector.

According to a third aspect of the present invention there is provided for a method of screening a mixed ecology sample comprising a plurality of microalgae species for microalgae having the ability to be transfected by a vector, the method comprising (i) providing the matrix-mediated cell culture system of the first aspect as described herein; (ii) incubating the immobilised microalgal cells with the vector; and (iii) detecting expression of the heterologous polypeptide of interest, wherein expression of the heterologous polypeptide of interest is indicative of the ability of the species of microalgae to be transfected.

In a first embodiment of the method of screening a mixed ecology sample the mixed ecology sample further comprises spores, bacteria, zooplankton and/or macroalgae.

According to a further embodiment of the third aspect, the method further comprises a step of removing medium from the immobilised microalgal cells prior to the step of incubating the immobilised microalgal cells with the vector.

In a further embodiment of the third aspect the method of screening a mixed ecology sample further comprises a step of cell sorting by flow cytometry.

In yet another embodiment of the method of screening a mixed ecology sample, the method comprises a step of selecting for heterotrophic microalgal cells by growing the mixed ecology sample in darkness prior to incubating the immobilised microalgal cells with the vector.

According to a fourth aspect of the present invention there is provided for a method of producing a heterologous polypeptide of interest in a microalgal cell, the method comprising (i) providing the matrix-mediated cell culture system of the first aspect as described herein; (ii) incubating the immobilised microalgal cells with the vector; (iii) expressing the heterologous polypeptide of interest, including a pharmacological peptide; (iv) recovering the heterologous polypeptide of interest from the matrix-mediated cell culture system; and (v) purifying the heterologous polypeptide of interest. It will be appreciated by those of skill in the art that the microalgal cell culture may comprise an axenic sample comprising a single species of microalgae or a mixed ecology sample comprising a plurality of microalgae species.

According to a fifth aspect of the present invention there is provided for the use of a porous matrix, preferably comprising diatomaceous earth (SiO₂), in a cell culture system, wherein cells are immobilised on the porous matrix, and further wherein the immobilisation of the cells on the porous matrix allows for filtration of a liquid medium out of the cell culture and results in the formation of interstitial spaces between the cells, thereby protecting the cells from mechanical stress, such as shearing from vacuuming.

In one embodiment of this aspect the cells may be microalgal cells or plant cells, or other cells in culture.

According to a sixth aspect of the present invention there is provided a method of harvesting endogenous compounds, proteins and/or metabolites from microalgal cells in a liquid growth medium, the method comprising (i) providing a porous matrix; (ii) immobilising a microalgal cell culture comprising microalgal cells on the porous matrix; (iii) optionally removing the immobilised microalgal cells from the medium; and (iv) recovering the endogenous compounds, proteins and/or metabolites from the microalgal cells or the medium.

According to a further embodiment of this aspect of the invention the endogenous compounds, proteins and/or metabolites may be exported or secreted from the microalgal cell into the medium and recovered from the medium. It will be appreciated that the endogenous compounds, proteins and/or metabolites may be recovered from the microalgal cells or the medium by passing a solvent through the microalgal cell culture.

According to a further aspect of the present invention there is provided for a method of harvesting low-density or high-density algal biomass from a liquid growth medium and/or for recovering endogenous compounds, proteins and/or metabolites from microalgal cells, the method comprising (i) providing a porous matrix as described herein; (ii) immobilising a microalgal cell culture comprising microalgal cells on the porous matrix; (iii) removing the immobilised microalgal cells from the medium; (iv) separating low-density algal biomass for high-density algal biomass; and/or (iv) recovering endogenous compounds, proteins and/or metabolites from the microalgal cells.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Non-limiting embodiments of the invention will now be described by way of example only and with reference to the following figures:

FIG. 1: An electron micrograph of diatomaceous earth (SigmaAldrich®), a silicious oxide of natural origin that is inert and insoluble in aqueous solvents.

FIG. 2: A schematic diagram of the pTrakc ERH::rfp vector. This vector not only allows for the production of two heterologous proteins, the first is the fluorescent protein, ref fluorescent protein (rfp), the second is the enzyme, neomycin phosphotransferase II (nptII), which allows for antibiotic selection of stably transfected cell lines.

FIG. 3: A schematic diagram of the pTrac vector. This vector is similar to the pTrakc ERH::rfp vector except it does not introduce nptII. This vector is used for transient expression of a protein of interest, but may be susceptible to cellular silencing as only one gene copy is present.

FIG. 4: A schematic diagram of the pRic vector. This vector self-replicates inside transfected cells, so increasing gene copy number, overcoming various cellular silencing mechanisms, which may greatly increase heterologous protein yields.

FIG. 5: Antibiotic resistance acquisition to G-418 by susceptible algal isolate, Ankystrodesmus gracialis and nptII gene detection via PCR.

FIG. 6: Non-heterotrophic microalgal species after 3 weeks of maintenance in the diatomaceous earth-mediated matrix, grown on a glucose-supplemented minimal salt medium.

FIG. 7: Heterotrophic microalgal species after 3 weeks of maintenance in the diatomaceous earth-mediated matrix, grown on a glucose-supplemented minimal salt medium.

FIG. 8: A—Heterotrophic vs mixotrophic growth of C. vulgaris; with and without Kanamycin; B—Mixotrophic vs autotrophic growth of three South African isolates; C—Cryogenically revived heterotrophic isolates.

FIG. 9: A—Chlorella vulgaris UTEX 395 column survival with and without agrobacterium (LBA4404); B—Chlorella vulgaris UTEX 395 column harvest efficiency with constant Celite:algal biomass, but increasing total volume.

FIG. 10: Heterotrophic and A. tumefaciens-transfectable microalgal species, shown producing Horseradish Peroxidase (HRP), before and after ELISA detection assay for HRP. Ptrac_ indicates the negative control, where microalgal cell population was transfected with an A. tumefaciens strain containing the Ti plasmid, but without exogenous gene. pTRAc::HRP contains the HRP gene under the control of a singly-inserting Ti plasmid. pRIC::HRP is a Ti plasmid that inserts a lone gene copy of the HRP gene, though this then self-replicates within the cell through rolling-loop replication.

FIG. 11: Following matrix-assisted cell pack-mediated transformation with the various vectors, the cell packs were resuspended in liquid growth medium. It can be seen that both HRP-expressing vectors had a pronounced effect on microalgal cell numbers.

FIG. 12: Western Blot demonstrating the production of HRP in two biological cell-pack repeats. Lane 1 contains Colour Protein Standard Broad Range Marker (New England BioLabs. P7712S). Lane 2 contains the negative control—this was an algal cell-pack transfected with pTRAc::eGFP. Lane 3 contains an HRP standard. Lanes 4 and 5 contain an algal cell-pack transfected with pTRAc::HRPΔC.

FIG. 13: Primary antibody: anti-GFP produced in rabbit. Secondary antibody: anti-rabbit alkaline phosphatase conjugate. Lane 1: GFP positive control (+−26.9 kDa). Lane 2: −ve(GFP): algal isolate MPA16.1 (Desmodesmus sp.) exposed to vector. Lane 3: t1: algal isolate MPA16.1 (Desmodesmus sp.) exposed to vector containing eGFP. Arrow denotes corresponding band.

FIG. 14: Staining confirms that Chlorella vulgaris UTEX 395 grown heterotrophically was transiently transformed by with A. tumefaciens LBA4404 and pCAMBIA 1301 expressing GUS (pCAMBIA 1301::GUS): Panel A shows the negative control; Panel B1 shows GUS expression under microscope; Panel B2 is a photograph showing GUS expression of transformed algal cells; and Panel B3 shows GUS expression of the algal cells under microscope at higher magnification.

FIG. 15: Schematic representation of the matrix-mediated cell culture system of the present invention and methods of screening a species of microalgae or a mixed ecology sample for microalgae having the ability to be transfected by a vector or methods of producing a heterologous polypeptide of interest using the matrix-mediated cell culture system of the present invention.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items.

The present invention relates to the generation and cultivation of a cell pack using small cells of any description that cannot form a non-tissue mediated cell pack in the conventional manner. These cells may include microalgae and/or any small cell as well as a matrix-generating substance, such as diatomaceous earth (Celite®) or any similar microsphere (natural or synthetic) that can fulfil the same role in the system. This matrix-assisted cell pack allows for a high efficiency of genetic manipulation of the cell population as well as ease of screening following genetic manipulation, which is not possible according to the current art.

The applications of this invention include high throughput screening of cells, especially algal isolates or species for their ability to produce heterologous proteins. This allows for a combinatorial approach to assess which isolate is best suited to produce transgenic proteins when exposed to a transformation vector.

The present invention further allows for screening of environmental samples, containing multiple species, for example all microalgae from an ecology, simultaneously, for their ability to be genetically modified and to produce heterologous proteins. This application, does not require separation of the ecology into separate isolates, and allows for even more efficient screening of suitable species for their ability to be used as industrial microorganisms for heterologous protein production.

Further, once suitable species for protein production have been identified using the methods of the present invention, high genetic transformation rates of the cell population allow for the production of large quantities of heterologous protein on an industrial scale.

It has previously been demonstrated that through the removal of most of the liquid in a plant cell culture, non-tissue multilayered plant cell packs can be formed. Such plant cell packs allow for synthetic plant cells to be maintained in this state due to the cell pack environment consisting of a low-liquid, high-humidity environment that allows for media replacement and waste removal, whilst also allowing the cells to respire via interstitial airspaces. Additionally, these interstitial airspaces can be transiently replaced, for example via vacuum, by various reagents and/or biologics suspended or dissolved in aqueous media, which allows for maximal contact between the plant cells and the active substance(s). For instance, it has been found that when Agrobacterium tumefaciens, a genetically modified plant pathogen able to pass on genetic material to plants, was aspirated though a plant cell pack, cells in the cell pack were genetically modified with an efficiency of 80%. This is significant in comparison to liquid co-culture of plant cells and bacteria, where only around 5% of the plant cells were modified. The technique therefore allows for various applications utilizing plant cells for the efficient production of heterologous proteins and/or as biological columns for various biological assays. However, plant cell-packs are highly inefficient when larger pack volumes are used and are thus inadequate for larger scale production of heterologous proteins. Further, existing plant cell cultures and plant cell packs are based on plant cells which originate from whole higher plants and are in a derived state.

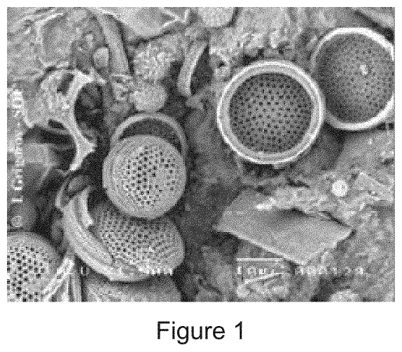

The present invention allows specifically for microalgae to be placed in porous columns for high throughput screening purposes or for industrial heterologous protein production, once suitable species have been identified. This is done via microalgal incorporation into a porous matrix made of diatomaceous earth, or any porous matrix-forming non-soluble substance (FIG. 1).

According to one embodiment of the present invention a single axenic microalgal culture, or a multi-species microalgal ecology derived from the environment or from mixed culture, is mixed with diatomaceous earth, excess media is removed with a vacuum, by suction, or by gravity.

This has the following advantages: First, microalgal cells are suspended in the diatomaceous earth matrix, which provides a high humidity environment but also, through interstitial airspace formation, allows for good rates of gaseous mass transfer to occur, so allowing for algal cell survival. Secondly, cell-pack formation allows for high levels of contacting between immobilised microalgae and A. tumefaciens or another gene transfer agent, thus maximising gene transfer from A. tumefaciens to the microalgae. Thirdly, as the matrix is permeable, new media can be vacuumed or suctioned (although gravity may be sufficient) through the algal matrix, so allowing for nutrient replenishment for microalgal survival. As new nutrients are introduced into the cell-pack, so old media can be removed, with the simultaneous removal of any soluble secreted heterologous protein products of interest, following cellular export from algal cells. In addition, should protein products be retained intracellularly, algal cell packs can be resuspended with relative ease, so that, once the diatomaceous earth has been allowed to sediment out of suspension, transfected algal cells can be removed with resuspension media, dewatered and processed.

The microalgal cell packs of the present invention allow for a number of applications due to their unique properties. Firstly, many separate axenic algal cultures can be individually assessed simultaneously for their ability to be genetically modified with A. tumefaciens. Here separate algal cell packs are created for each axenic algal culture, where each isolate contains only one species of algae and contains no bacterial or fungal contaminants. Each algal cell pack is then exposed to the genetically modifying vector via vacuum or suction infiltration of the specific A. tumefaciens strain through each cell pack.

Secondly, once a suitable algal isolate has been identified, various vectors and parameters can easily be optimised for maximisation of heterologous protein production.

Thirdly, microalgal species (or even whole microalgal ecologies) can be simultaneously exposed to a genetically modifying agent such as Agrobacterium spp., and only those that exhibit traits that are necessary and useful for industrial application, for example high expression rates of heterologous peptides of interest, are then retained. Of note here is that microalgae are the most biodiverse eukaryotic organisms. Thus, screening for specific useful species out of the possible four million proposed species must be accomplished in a very efficient manner, where individual species demonstrate a combination of several traits relevant to industrial heterologous protein production. Such traits include the ability to be transfected by A. tumefaciens, where, for instance, several microalgal isolates are exposed to an A. tumefaciens strain simultaneously. This transfection then induces fluorescence and/or chemical resistance to an algal toxin in only those microalgal species that are suitable to the algal cell-pack transfection process, allowing for their corresponding selection and retention.

Further, cell packs can be upscaled for production, so as to allow for maximal gene transfer to algal biomass cultured previously and, depending on whether heterologous protein products are exported or are retained intracellularly, wash-through harvesting of the protein product or cell separation from the matrix, dewatering and cell lysis may be performed, followed by appropriate downstream purification of the protein product. This is of special interest to cost minimisation when producing heterologous peptides of interest, as algal biomass can be cultured, not in complete isolation as is legislatively required for genetically modified organisms (GMO), but rather, the unmodified ‘wild-type’ is cultured to the desired biomass in ponds separate to the processing and transfection facility. This biomass is then processed and converted into heterologous protein only once inside the transfection facility after column formation and dewatering under vacuum. This not only allows for a far smaller industrial footprint, but also ameliorates the need for large media volumes as well as the associated costs of maintaining selection with various high-cost antibiotics. Higher resultant concentrations may decrease downstream processing costs.

To detect whether a microalgal species can produce heterologous protein, a large proportion of the population must be transfected. For this, a cell pack-type approach is required, but this is not possible by currently known processes, due to the relatively small size of microalgal cells. For this reason, microalgae require a matrix that is porous, allows for interstitial airspace formation, and allows for A. tumefaciens-mediated gene transfer to occur. This invention relates specifically to this process and specifically the use of a porous, inert matrix-forming agent such as diatomaceous earth in stabilising the cell packs of the present invention.

As used herein, “microalgae” refers to unicellular organisms which exist individually, or which occur in chains or groups and which are capable of performing photosynthesis, although some algal species are also capable of growing heterotrophically. Depending on the species, microalgae sizes can range from a few micrometers (μm) to a few hundred micrometers. Unlike higher plants, microalgae do not have specialised cells, tissues and organs, such as roots, stems, or leaves. The terms “microalgae” and “algae” are used interchangeably herein to refer to microalgae. Further, the term “microalgal cells” refer to cells of the microalgae. Examples of suitable microalgae include Anabaena spp., Chlamydomonas spp., Chlorella spp., Desmodesmus spp., Dunaliella spp., Nannochloropsis spp., Phaeodactylum spp., Porphyridium spp., Scenedesmus spp., Spirulina spp., Synechoccus spp., and Thalassiosira spp. For example, Chlorella vulgaris may suitably be used.

As used herein, the term “plant” refers to “higher plants”, being multicellular members of the taxonomical kingdom Plantae having specialised cells arranged into tissues and/or organs, such as roots, stems, or leaves.

As used herein, the term “matrix-mediated cell pack” or “matrix-assisted cell pack” refers to a cell column/pack that consists of a cell suspension and a porous matrix, for example diatomaceous earth, which stabilises the cells. It will be appreciated by those of skill in the art that other suitable porous matrix materials may be used, including Perlite, synthetic SiO₂, and/or any other inert microsphere (natural or synthetic) that can fulfil the same role.

As used herein, the term “non-tissue mediated cell pack” refers to a cell column/pack that consists only of cells, with no added matrix, wherein the cell suspension culture is passed through a filter, under vacuum, separating the liquid media (as flow-through) from the cells (as filtrate).

The term “heterologous polypeptide of interest” or “heterologous protein” as used herein refers to any polypeptide that does not occur naturally in an algal cell. A heterologous polypeptide of interest may thus include protozoal, bacterial, viral, fungal or animal proteins. The heterologous polypeptide of interest is intended for expression in an algal cell using the methods of the present invention. Non-limiting examples of heterologous polypeptides of interest may include reporter polypeptides, pharmacological polypeptides (e.g., for medical uses, for cell- and tissue culture) or industrial polypeptides (e.g. enzymes, growth factors) that can be produced according to the methods present invention. The term “protein” should be read to include “peptide” and “polypeptide” and vice versa. It will be appreciated that a “reporter polypeptide” may be any polypeptide or protein whose transcription, translation and/or post-translation activity can be detected. Examples of reporter polypeptides include, but are not limited to, luciferase, alkaline phosphatase, green fluorescent protein, beta-galactosidase, horse radish peroxidase, and the like. The expression of the reporter polypeptide is used in the present invention as an indicator of the transformation of the microalgal cells.

As used herein the term “transformed algae” or “transfected algae” both refer to algae or an algal cell which has either been stably or transiently transformed or transfected in order to express a heterologous polypeptide or which has been infiltrated with at least one expression vector which transiently expresses a heterologous polypeptide in the algae or an algal cell. In a first aspect of the invention, the algal cell pack of the present invention allows for transient algal heterologous protein production the first time the algal cells are transfected. According to a second aspect of the invention, once the Agrobacterium spp. has been passed through the cell pack, the cells may be resuspended in an antibiotic selection media, thereby selecting for stably transformed algal cells.

The term “purified”, relates to the isolation of a molecule or compound in a form that is substantially free of contamination or contaminants. Contaminants are normally associated with the molecule or compound in a natural or cultured environment, purified thus means having an increase in purity as a result of being separated from the other components of an original composition or culture.

The term “isolated”, is used herein and means having been removed from its natural environment.

The terms “nucleic acid”, “nucleic acid molecule” and “polynucleotide” are used herein interchangeably and encompass both ribonucelotides (RNA) and deoxyribonucleotides (DNA), including cDNA, genomic DNA, and synthetic DNA. The nucleic acid may be double-stranded or single-stranded. Where the nucleic acid is single-stranded, the nucleic acid may be the sense strand or the antisense strand. A nucleic acid molecule may be any chain of two or more covalently bonded nucleotides, including naturally occurring or non-naturally occurring nucleotides, or nucleotide analogs or derivatives. The term “DNA” refers to a sequence of two or more covalently bonded, naturally occurring or modified deoxyribonucleotides.

As used herein, the term “gene” refers to a nucleic acid that encodes a functional product, for instance a RNA, polypeptide or protein. A gene may include regulatory sequences upstream or downstream of the sequence encoding the functional product.

As used herein, the term “coding sequence” refers to a nucleic acid sequence that encodes a specific amino acid sequence. On the other hand, a “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

In some embodiments, the genes used in the method of the invention may be operably linked to other sequences. By “operably linked” is meant that the nucleic acid molecules encoding the recombinant polypeptides of the invention and regulatory sequences are connected in such a way as to permit expression of the proteins when the appropriate molecules are bound to the regulatory sequences. Such operably linked sequences may be contained in vectors or expression constructs which can be transfected into host cells for expression. It will be appreciated that any vector or vectors can be used for the purposes of expressing the recombinant antigenic polypeptides of the invention.

The term “promoter” refers to a DNA sequence that is capable of controlling the expression of a nucleic acid coding sequence or functional RNA. A promoter may be based entirely on a native gene or it may be comprised of different elements from different promoters found in nature. Different promoters are capable of directing the expression of a gene in different cell types, or at different stages of development, or in response to different environmental or physiological conditions. A “constitutive promoter” is a promoter that direct the expression of a gene of interest in most host cell types most of the time.

The term “recombinant” means that something has been recombined. When used with reference to a nucleic acid construct the term refers to a molecule that comprises nucleic acid sequences that are joined together or produced by means of molecular biological techniques. The term “recombinant” when used in reference to a protein or a polypeptide refers to a protein or polypeptide molecule which is expressed from a recombinant nucleic acid construct created by means of molecular biological techniques. Recombinant nucleic acid constructs may include a nucleotide sequence which is ligated to, or is manipulated to become ligated to, a nucleic acid sequence to which it is not ligated in nature, or to which it is ligated at a different location in nature. Accordingly, a recombinant nucleic acid construct indicates that the nucleic acid molecule has been manipulated using genetic engineering, i.e. by human intervention. Recombinant nucleic acid constructs may be introduced into a host cell by transformation. Such recombinant nucleic acid constructs may include sequences derived from the same host cell species or from different host cell species.

The term “vector” refers to a means by which polynucleotides or gene sequences can be introduced into a cell. There are various types of vectors known in the art including plasmids, viruses, bacteriophages and cosmids. Generally, polynucleotides or gene sequences are introduced into a vector by means of a cassette. The term “cassette” refers to a polynucleotide or gene sequence that is expressed from a vector, for example, polynucleotide or gene sequences encoding heterologous polypeptides. A cassette generally comprises a gene sequence inserted into a vector, which in some embodiments, provides regulatory sequences for expressing the polynucleotide or gene sequences. In other embodiments, the vector provides the regulatory sequences for the expression of the heterologous polypeptides. In further embodiments, the vector provides some regulatory sequences and the nucleotide or gene sequence provides other regulatory sequences. In a further embodiment of the present invention, the vector is a broadly transforming bacteria-associated vector, preferably an Agrobacterium spp. vector.

The following examples are offered by way of illustration and not by way of limitation.

### Example 1

Stable Gene Expression in the Microalgae Ankystrodesmus Gracialis, Following Algal Cell Pack-Mediated Transfection with A. tumefaciens

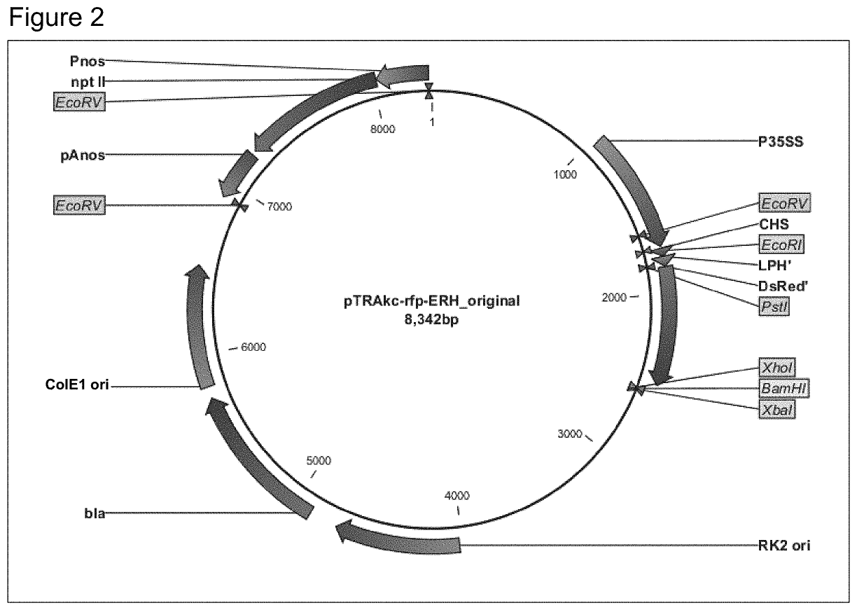

To demonstrate transfection, the vector pTRAkc ERH::rfp was used (FIG. 2). This vector not only inserts the gene, Red Fluorescent Protein (rfp) into host cells, in this case microalgae, but also inserts the resistance gene, neomycin phosphotransferase II (nptII). NptII allows for the production of an enzyme able to degrade various antibiotics, such as Kanamycin as well as G-418. For this reason several isolates of green microalgae were assessed for G-418 susceptibility. To this end, it was found that A. gracialis was susceptible to G-418 at 20 mg·L⁻¹, exhibiting complete growth inhibition. A. gracialis was additionally able to withstand exposure to 200 mg·L⁻¹ Cefotaxime, which is fundamental for the selective removal of possible A. tumefaciens contamination post transfection.

Diatomaceous earth-mediated algal cell packs were created as follows: diatomaceous earth (Celite® 545, Merck) was mixed into axenic A. gracialis culture, so that 0.75 g of diatomaceous earth was used per 5 ml of algal culture. Thereafter, 5 ml of algal-diatomaceous earth suspension mixture was pipetted into clear plastic columns (Bio-Rad Laboratories, Inc), followed by excess media removal with vacuum.

Columns were then transfected with A. tumefaciens strain GV 3101 MpRK90 harbouring pTRAkc ERH::rfp at an OD₆₀₀ of 3.4 and left to incubate for an hour before excess A. tumefaciens was introduced, so reinitiating interstitial airspaces. Simultaneously, A. gracialis diatomaceous earth columns were formed and transfected with no vector, acting as a negative control.

Three days following initial transfection, both transfected and non-transfected columns were harvested as follows: algal-diatomaceous earth was removed to sterile 15 ml falcon tubes containing 15 ml ddH₂O (autoclaved and filter sterilised, 0.22 μm) followed by vortexing. Diatomaceous earth was the allowed to settle for 15 min. Algal suspension was then removed to 2 ml eppendorf tubes and a cell count was performed.

Samples were then diluted accordingly and 200 μL containing 3×10⁶ cells of each sample were then plated onto heterotrophic media plates (2% bacteriological agar containing: 3 N Bold's Basal Media with 20 mM Glucose and Glycerol as well as 20 mg·L⁻¹ G-418 and 200 mg·L⁻¹ Cefotaxime).

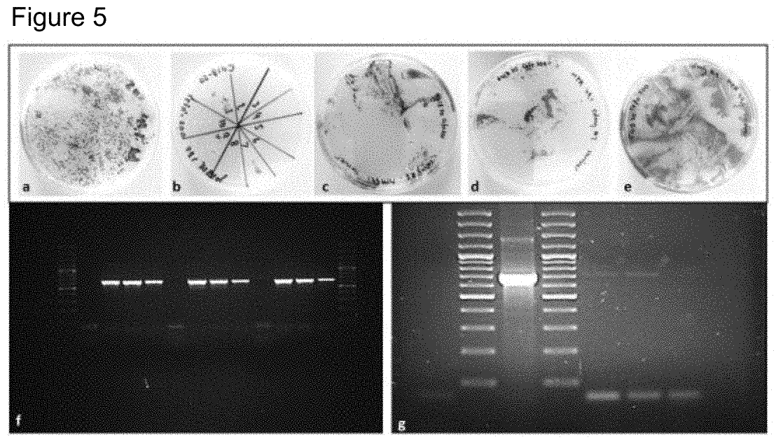

Plates were allowed to incubate under lights (12:12; light:dark) until colony formation occurred. No colonies formed on selection plates containing non-transfected A. gracialis, indicating total inhibition of wild-type growth by G-418 (data not shown). However, on selection plates containing transfected A. gracialis in excess of a thousand colonies formed (FIG. 5a).

From this initial plate, ten colonies were picked at random and plated onto a second selection plate (FIG. 5b). Here, three of the picked colonies maintained G-418 resistance and the ability to propagate in the presence of 20 mg·L⁻¹ G-418. These were then picked and diluted in ddH₂O and plated onto separate plates (FIGS. 5c, 5d and 5e). Interestingly, the last colony picked demonstrated a marked increase in growth-ability relative to the other two colonies (FIG. 5e).

Colony polymerase chain reaction (PCR) was then done to amplify nptII to confirm that G-418 resistance in the above was indeed due to transfection of nptII.

This was done from plate scrapings of the three plates shown in FIGS. 5c, 5d and 5e as follows: algal cells were collected from each plate and removed to sterile PCR tubes containing 50 μl 5% Chelex suspension. These were vortexed for 1 min, boiled at 98° C. for 5 min and then cooled on ice for 1 min, then vortexed for 1 min and centrifuged at 13000 rpm for 1 min.

PCR conditions were as follows:

| PCR | | |
|---|---|---|
| primers | Primer Sequence | SEQ ID NO |
| FWD | GAGGCTATTCGGCTATGACTG | SEQ ID NO: 1 |
| REV | ATCGGGAGCGGCGATACCGTA | SEQ ID NO: 2 |

PCR reaction: 95° C. for 3 min (one cycle), 94° C.
for 1 min, 55° C. for 1 min, 72° C. for 1 min 30
sec (40 cycles), 72° C. for 10 min (one cycle)
and then held at 4° C.

### Example 2

Transient Heterologous Protein Production of the Industrial Enzyme Horseradish Peroxidase (HRP)

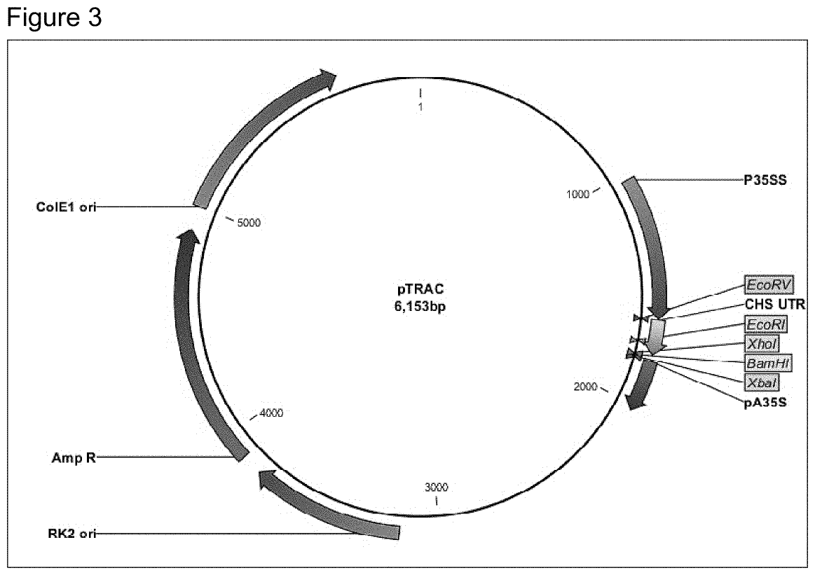

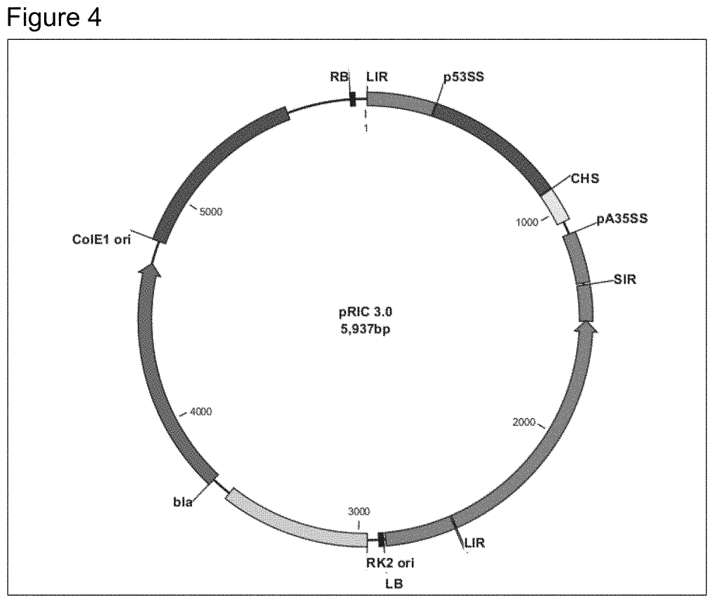

Twenty axenic algal species were isolated from various inland lakes around South Africa's Mpumalanga region. These were grown up in 15 ml of a combination medium (3:1, 3N Bold's Basal Medium (BBM): Lysogeny Broth (LB)) until stationary phase was reached in 50 ml shake flasks. Four days prior to cell pack formation, 500 μl LB broth was added to each shake flask. At this stage, three A. tumefaciens strains: (1) GV3101 pMp90RK pTRAc::HRPdeltaC; (2) GV3101 pMp90RK pRIC::HRPdeltaC and (3) GV3101 pMp90RK pTRAc::_ were grown up overnight. The first strain introduces a single copy of a gene derived from the horseradish Armoracia rusticana, known as horseradish peroxidase (HRP). The second introduces a self-replicating viral based construct that copies its own DNA intracellularly, so providing more HRP gene copies for protein production (FIG. 4), while the third introduces a vector containing no gene, acting as a negative control (FIG. 3). The HRP gene, either in the native horseradish or in transgenic organisms produces the industrially relevant enzyme, horseradish peroxidase, which has several diagnostic and therapeutic applications with a retail price of approximately 370 GBP per 100 mg.

Cell packs were formed as follows: Diatomaceous earth was mixed into each algal culture, so that 0.75 g of diatomaceous earth was used per 5 ml of algal culture. Five ml of algal-diatomaceous earth mixture was then pipetted into clear plastic columns (BioRad), followed by excess media removal with vacuum. Each isolate was then transfected with each of the A. tumefaciens strains, such that 1 ml (0D600=0.2) of each was vacuumed into the cell pack and left to incubate for an hour before removal of liquid from the cell packs to reform interstitial air spaces.

The following day 500 μl of 3 N BBM with 4 g·l⁻¹ glucose and MES, was vacuumed through each cell pack. This was followed the next day by collection of any liquid into 2 ml Eppendorf tubes, which were assayed for horseradish peroxidase activity (TMB Microwell Peroxidase Substrate System, KPL, Gaitherburg Md. 20878, USA) which was done on days 3, 5 and 7.

In addition to this, the isolate showing the greatest colour change using the HRP assay was harvested and analysed by a western blot to allow for HRP detection. This was done as follows: After 7 days post transfection, algal cell-packs were resuspended in 10 ml PBS buffer with vortexing. This dissociated algal biomass from the Celite® matrix. Celite® was then allowed to sediment for 30 min, and algal PBS suspension was then removed to a 15 ml centrifuge tube. This was then centrifuged for 10 minutes at 10 000 rpm. Supernatant was poured off and pellet was resuspended in 1 ml PBS and placed in Eppendorf tubes. These were then centrifuged at 13 000 rpm for 3 min and supernatant was removed. Algal pellet was then frozen with liquid nitrogen and cells lysed with a micropestle. This was followed by the addition of 150 μl PBS to cell lysate. This was then centrifuged for 3 minutes at 13 000 rpm. 100 μl of supernatant was then removed to another Eppendorf tube to which 25 μl of Sample Application Buffer was added. Samples were then denatured at 94° C. for 5 minutes and run on a 2% SDS-PAGE gel. Following nitrocellulose transfer and immobilisation, blots were incubated over night with primary anti-HRP antibody (ABCam ab2110: anti-HRP produced in mice; 1:5000 concentration). Primary antibody was then detected with anti-mouse alkaline phosphatase conjugated secondary antibody produced in goat.

Each cell pack was maintained in this manner, and three weeks after initial A. tumefaciens transfection, the highest producing isolate was then resuspended in 3 N BBM media, where a plate count of viable cells was performed.

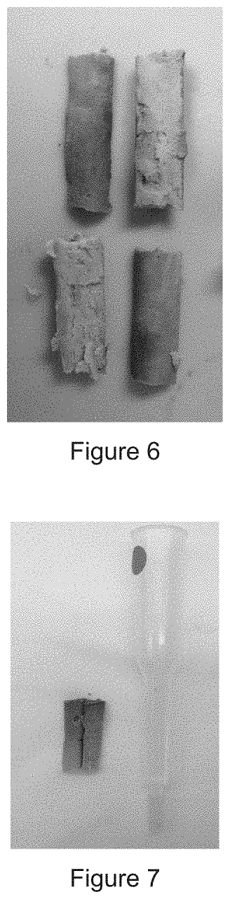

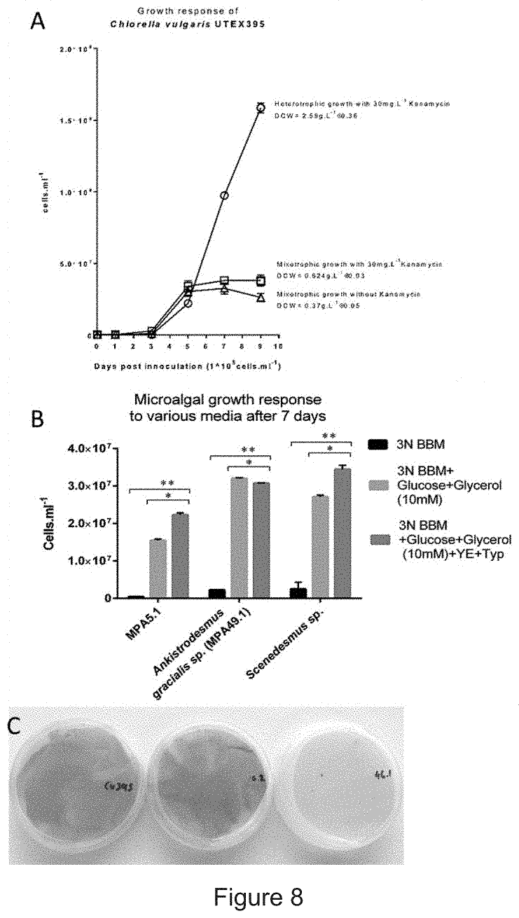

Algal species that are not able to reproduce heterotrophically in the presence of glucose are not able to form colonies on the inside of the cell packs, which are light-impermeable (FIG. 6), while those that can alter their metabolism to allow for heterotrophic glucose respiration are able to colonise the inside of the cell pack (FIG. 7). Further, heterotrophic metabolism is more productive. In addition, it is useful for protein production if the microalgae have the ability to be stored at very low temperatures, halting the cell cycle, and then later revived. This enables the halting of strain evolution and prevents batch-to-batch variation. However, cryogenic preservation and heterotrophic growth are disparate abilities that may not be shared by a single isolate. Thus, a model microalgae (Chlorella vulgaris UTEX 395) was assessed for its ability to withstand cryogenesis and grow heterotophically. The isolate was grown heterotrophically to assess media requirements for heterotrophic metabolism as well as initial growth investigations via cell counts. An axenic algal library, stored at −80°, was then revived on solid heterotrophic media (in the dark). Chlorella vulgaris was successfully grown heterotrophically completely in the dark. From the initial library, thirteen isolates were cryogenically revived on autotrophic media and five were revived on heterotrophic media directly. The isolates were then investigated for demonstrated high biomass yields when grown heterotrophically. Interestingly, biomass productivity was affected by the addition of amino acids in a strain specific manner (FIG. 8).

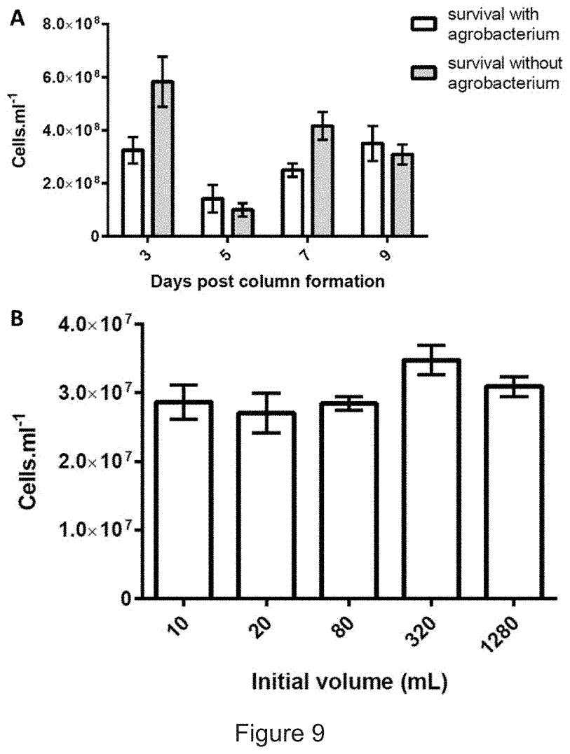

Algal survival in a column was investigated, both with and without A. tumefaciens, after feeding with an enriched media that the specific isolate was selected for, to allow heterotrophic growth in the absence of light. Thereafter, harvest efficiency was determined, where 10 ml of a dense, heterotrophically grown, algal culture was mixed with 2 g of Celite®. The total volume was increased via dilution, which demonstrates the ability of the column to harvest high density, heterotrophic algal cultures, as well as low density autotrophic algal cultures using Celite®. Once column formation was completed, the same cell number was present, although the initial volume was highly variant (FIG. 9).

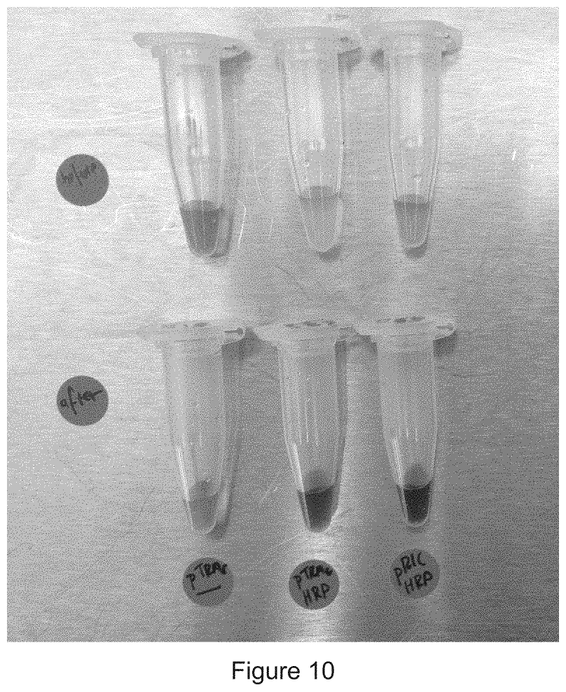

Twenty axenic isolates were used to form algal cell packs and transfected with A. tumefaciens strains that introduce the HRP gene and a positive reaction was detected through colour change with the KPL Peroxidase Substrate System in the isolates. Of note, one of the isolates demonstrated a marked reaction with both the single gene inserting vector (pTRAc::HRPΔC) and the self-replicating vector (pRIC::HRPΔC) (FIG. 10). This indicates that not only is HRP being produced by the transfected algal cell-pack but that the algae are exporting the HRP into the media.

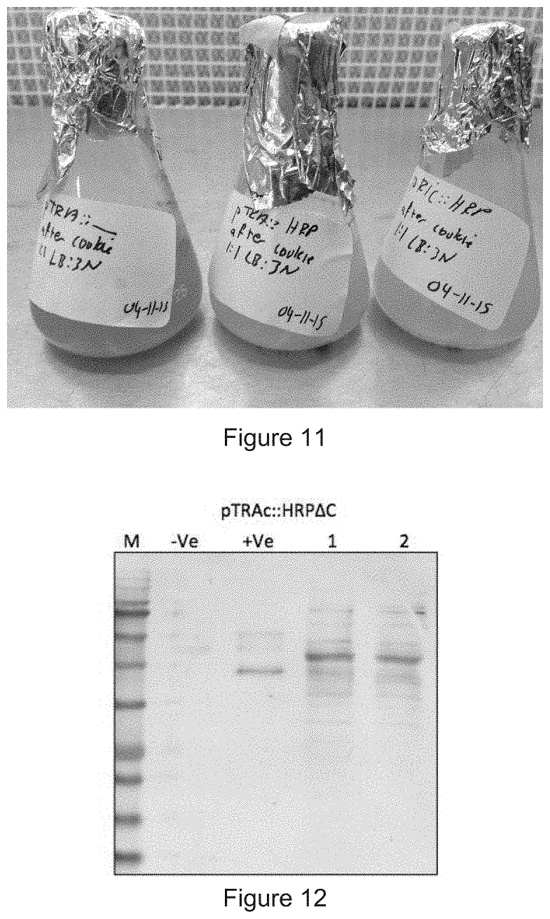

It can be seen that cells isolated from the cell-packs with HRP had their viability compromised in relation to those transfected with a vector not containing an HRP gene (FIG. 11).

Western blotting for the presence of HRP, a 43 kD protein, showed a positive result, not only by demonstrating monoclonal antibody binding in the two pTRAc biological replicates but also having the correct molecular size. The standard is smaller and thus lower down on the blot due to differential glycosylation patterning (FIG. 12).

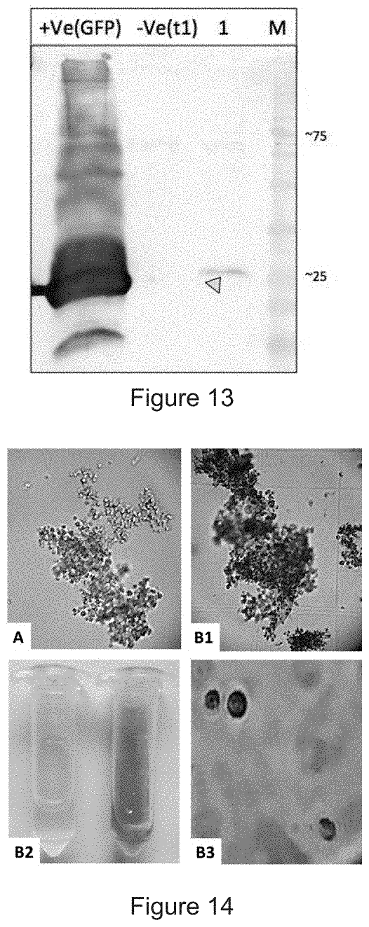

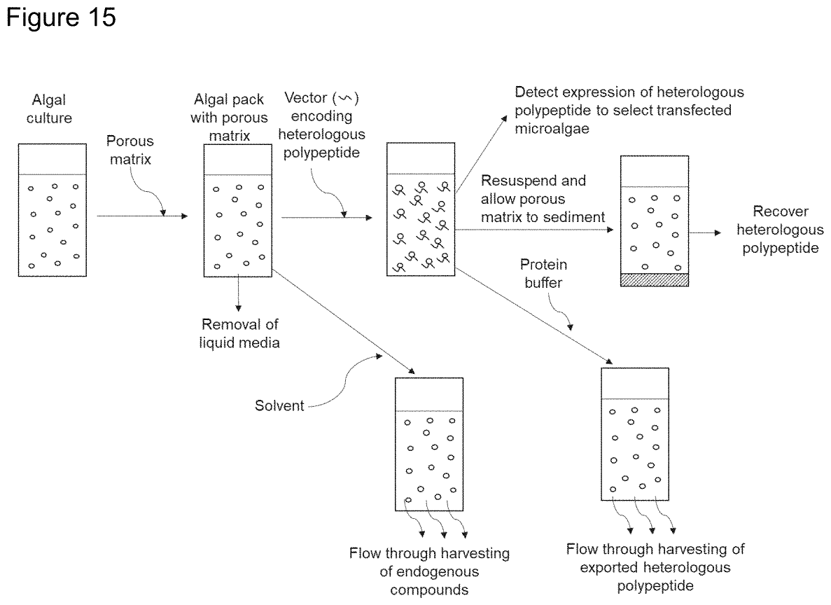

In a similar western-blot based analysis, but utilising Green Fluorescent Protein (GFP) as the heterologous protein instead of HRP, GFP was expressed using a chloroplast targeting vector (FIG. 13).

Further, Chlorella vulgaris UTEX 395 grown heterotrophically was transiently transformed by with A. tumefaciens LBA4404 and pCAMBIA 1301 expressing GUS (pCAMBIA1301::GUS) (FIG. 14).

1. A matrix-mediated cell culture system comprising:
(i) a porous matrix;
(ii) a microalgal cell culture comprising microalgal cells immobilised on the porous matrix; and
(iii) a vector including a nucleic acid sequence encoding a heterologous polypeptide of interest,
wherein the immobilisation of the microalgal cells on the porous matrix results in the formation of interstitial spaces between the microalgal cells to allow for increased contact of the microalgal cells with the vector compared with a culture of microalgal cells which are not immobilised on a porous matrix, thereby allowing the microalgal cells to be transfected with the vector.

2. The matrix-mediated cell culture system of claim 1, wherein the porous matrix comprises diatomaceous earth (SiO2).

3. The matrix-mediated cell culture system of claim 1, wherein the porous matrix has a particle size in the same size range as the microalgal cells.

4. The matrix-mediated cell culture system of claim 1, wherein the vector is an Agrobacterium spp. vector.

5. The matrix-mediated cell culture system of claim 1, wherein the heterologous polypeptide of interest is a reporter polypeptide.

6. The matrix-mediated cell culture system of claim 5, wherein the reporter polypeptide is selected from the group consisting of luciferase, alkaline phosphatase, green fluorescent protein, beta-galactosidase, and horse radish peroxidase.

7. The matrix-mediated cell culture system of claim 1, wherein the heterologous polypeptide of interest is a pharmacological polypeptide.

8. The matrix-mediated cell culture system of claim 1, wherein the matrix-mediated cell culture system is medium-deprived.

9. The matrix-mediated cell culture system of claim 1, wherein the immobilisation of the microalgal cells on the porous matrix is by non-covalent adhesion of the microalgal cells to the porous matrix.

10. The matrix-mediated cell culture system of claim 1, wherein the microalgal cell culture comprises an axenic sample comprising a single species of microalgae.

11. The matrix-mediated cell culture system of claim 1, wherein the microalgal cell culture comprises a mixed ecology sample comprising a plurality of microalgae species.

12. The matrix-mediated cell culture system of claim 11, wherein the mixed ecology sample further comprises spores, bacteria, zooplankton and/or macroalgae.

13. A method of screening a species of microalgae for its ability to be transfected by a vector, the method comprising:
(i) providing the matrix-mediated cell culture system of claim 1;
(ii) incubating the immobilised microalgal cells with the vector;
(iii) transfecting the microalgal cells with the vector; and
(iv) detecting expression of the heterologous polypeptide of interest, wherein expression of the heterologous polypeptide of interest is indicative of the ability of the species of microalgae to be transfected.

14. The method of claim 13, wherein the microalgal cell culture comprises an axenic sample comprising a single species of microalgae.

15. The method of claim 13, wherein the method further comprises a step of removing medium from the immobilised microalgal cells prior to the step of incubating the immobilised microalgal cells with the vector.

16. A method of screening a mixed ecology sample comprising a plurality of microalgae species for microalgae having the ability to be transfected by a vector, the method comprising:
(i) providing the matrix-mediated cell culture system of claim 1;
(ii) incubating the immobilised microalgal cells with the vector; and
(iii) detecting expression of the heterologous polypeptide of interest, wherein expression of the heterologous polypeptide of interest is indicative of the ability of the species of microalgae to be transfected.

17. The method of claim 16, wherein the mixed ecology sample further comprises spores, bacteria, zooplankton and/or macroalgae.

18. The method of claim 16, further comprising a step of removing medium from the immobilised microalgal cells prior to the step of incubating the immobilised microalgal cells with the vector.

19. The method of claim 16, further comprising a step of cell sorting by flow cytometry.

20. The method of claim 16, further comprising a step of selecting for heterotrophic microalgal cells by growing the mixed ecology sample in darkness prior to incubating the immobilised microalgal cells with the vector.

21. A method of producing a heterologous polypeptide of interest in a microalgal cell comprising:
(i) providing the matrix-mediated cell culture system of claim 1;
(ii) incubating the immobilised microalgal cells with the vector;
(iii) expressing the heterologous polypeptide of interest;
(iv) recovering the heterologous polypeptide of interest from the matrix-mediated cell culture system; and
(v) purifying the heterologous polypeptide of interest.

22. The method of claim 21, wherein the microalgal cell culture comprises an axenic sample comprising a single species of microalgae.

23. The method of claim 21, wherein the microalgal cell culture comprises a mixed ecology sample comprising a plurality of microalgae species.

24. A method of harvesting endogenous compounds, proteins and/or metabolites from microalgal cells in a liquid growth medium, the method comprising:
(i) providing a porous matrix;
(ii) immobilising a microalgal cell culture comprising microalgal cells on the porous matrix;
(iii) optionally removing the immobilised microalgal cells from the medium;
(iv) recovering the endogenous compounds, proteins and/or metabolites from the microalgal cells.
